(19) United States
(12) Patent Application Publication
(10) Pub. No.: US 2025/0090728 A1
(43) Pub. Date: Mar. 20, 2025

(54) SILICONE MATERIAL FUNCTIONALIZED WITH COPPER NANOPARTICLES WHICH REDUCES BACTERIAL LOAD AND BIOFILM FORMATION

(71) Applicant: UNIVERSIDAD BERNARDO O'HIGGINS, Santiago (CL)

(72) Inventors: María Cristina PAREDES ESCOBAR, Santiago (CL); David Arturo MONTERO FORERO, Santiago (CL); Alejandro Felipe ESCOBAR ÁLVAREZ, Santiago (CL); Gabriel Nelson ABARCA ANJARÍ, Santiago (CL)

(21) Appl. No.: 18/724,511

(22) PCT Filed: Dec. 9, 2022
(86) PCT No.: PCT/CL2022/050126

§ 371 Date: Jun. 26, 2024

(30) Foreign Application Priority Data
Dec. 30, 2021 (CL) 3572-2021

(51) Int. Cl. A61L 29/02; A61L 29/08; A61L 29/16; B82Y 5/00; B82Y 40/00; C08L 71/02

(52) U.S. Cl. CPC A61L 29/02; A61L 29/085; A61L 29/16; C08L 71/02; A61L 2300/102; B82Y 5/00; B82Y 40/00

(57) ABSTRACT

The invention provides a silicone material functionalized with PEG-coated (PEGylated) copper nanoparticles that reduces bacterial load and biofilm formation. The material in accordance with the invention may be used in the manufacture of devices or products for use in the clinical and/or biomedical industry, food or any other industry where antibacterial and antibiofilm properties are desirable and necessary.

### FIELD OF THE INVENTION

The invention is part of the framework of materials science and technology at the service of society. Specifically, the present invention provides a silicone material functionalized with PEG-coated copper nanoparticles with antimicrobial and antibiofilm activity, so as to reduce the bacterial load and the development of biological films or biofilms of pathogenic bacteria on the surface of products or devices made with the material in accordance with the invention.

### BACKGROUND TO THE INVENTION

Microorganisms accumulate naturally on a wide variety of surfaces, where they form sedentary and sessile communities. These surfaces include domestic and industrial pipes, biocompatible materials, such as contact lenses, or medical devices, such as implants or urinary catheters, as well as plant and animal tissues, among many other surfaces. The accumulation of these aggregates of microorganisms in mono- or polymicrobial form are known as biological films or biofilms and can be composed of different bacterial and fungal communities.

On the other hand, silicone-based materials are increasingly used in various industries, as they have excellent heat resistance, dielectric properties, water repellency, flexible structure, as well as being biocompatible. In the case of cooking, for example, it is possible to use food-grade silicone in baking pans, mold coatings, and utensils. In the case of the pharmaceutical industry, it is possible to use medical devices made of silicone to deliver drugs with controlled release, and pharmaceutical-grade silicones can be used in molded devices such as intravaginal rings (IVRs) or intrauterine devices (IUDs) and in medicated implants, such as stents. Another frequent use of silicone in the biomedical industry is the manufacture of tubular devices such as cannulas, probes, and catheters.

Taking into account the versatility of the new uses of silicone and the fact that it is a surface that comes into contact with food, drugs and the body itself, it is necessary to have a material that prevents the formation of biofilms after the sustained use of articles and/or devices made with this material.

In particular, in the case of medical or clinical devices, healthcare-associated infections (HAIs) increase morbidity, mortality, and health care costs. Among them, catheter-associated urinary tract infection (CAUTI) is one of the most prevalent, where the use of an indwelling urinary catheter (used for more than 24 hours) is one of the main causes of bacteriuria and bacteremia. These infections remain uncontrolled despite improved clinical practices and the development of medical devices with antibacterial properties. Therefore, these infections are a current field of research that requires new control and prevention strategies.

The ideal material for the manufacture of a urinary catheter should be biocompatible, radiopaque, resistant to fouling and colonization by microorganisms, causing low discomfort, durable and affordable at a reasonable price. However, there is no urinary catheter on the market that meets all these requirements. The most commonly used materials in the manufacture of these devices are latex, silicone, polyurethane (PU) and polyvinyl chloride (PVC). Latex and PVC catheters are cheaper, but they can be quickly embedded and have a higher incidence of allergies. PU catheters have good biocompatibility, but their increased rigidity compared to silicone causes discomfort in some patients. Currently, the use of PU is more widespread in the manufacture of central venous catheters than in urinary catheters. In contrast, silicone is more suitable for permanent probing as it has high biocompatibility, absence of allergic responses, good chemical and thermal stability, and also a long shelf life (90 days). The main disadvantage of silicone is its higher cost relative to the other materials, and that it could be colonized by microorganisms.

There are currently a variety of urinary catheters that have been designed with the goal of reducing the risk of infection. These include catheters impregnated with antibiotics (nitrofural, minocycline, and rifampicin), silver oxide, and silver alloys. However, these catheters have little effect on the adhesion of uropathogens and in clinical trials have been effective in reducing the incidence of asymptomatic bacteriuria but not of CAUTI. More recent studies have evaluated the use of copper (CuNP) and silver (NpAg) nanoparticles to develop antimicrobial materials that can be used in medical devices. For example, Sehmi et al. (2015), embedded CuNP in polyurethane and silicone and showed that these polymers have an antimicrobial effect in vitro against Staphylococcus aureus and Escherichia coli. Similarly, Ballo et al. developed catheters impregnated with CuNP and NpAg that had antimicrobial activity in vitro but failed to prevent biofilm formation in vivo.

### STATE OF THE ART

In the previous art, various solutions are known that address the control or prevention of microbial growth and biofilm formation of pathogenic organisms in silicone materials and devices for medical use.

The patent U.S. Pat. No. 7,993,390B2 provides an implantable or insertable medical device that is resistant to microbial growth and biofilm formation, both in the device and in its environment. The devices described include a biocompatible matrix polymer region, an antimicrobial agent, and/or an inhibitor of biofilm formation. Among the alternatives developed, a polymer ureteral stent is shown where the bioactive agent is triclosan. Although it is the same technical problem, the solution used is different, as they use triclosan as an active compound that inhibits biofilm formation.

Sehmi, Sandeep K., et al. (2015) employs 2.5 nm copper nanoparticles as an antimicrobial compound in medical-grade silicone and polyurethane materials for in-hospital use. However, there is talk of encapsulation due to swelling and contraction, so in the case of silicone and considering the smaller size of copper nanoparticles, it is not the same type of material. In addition, the desired feature is to reduce microbial contamination on frequently handled surfaces in and around hospital wards, such as bed rails, tables, push plates, etc. The problem of preventing or reducing the formation of biofilm in the processed material is not addressed.

Iqbal, Zohora, et al. (2018) describes the effect of sterilization on modified silicone surfaces. It focuses on the stability of medical device biomaterials by considering all stages of preparation for surgery, including sterilization, so it explores the effect of five standard sterilization methods (autoclave, dry heat, hydrogen peroxide, ethylene oxide gas, and electron beam) on three surface coatings: PEG, pSBMA, and pMPC. The data shown suggest that autoclave and EtO treatments are suitable for PEG-silane.

Regmi, Amrit, et al. (2019) describes the formation of copper nanoparticles, made with two different concentrations of copper precursors by the coprecipitation method using NaBH₄ as a reducing agent and (PEG₆₀₀₀) as a stabilizer. The paper evaluates the antibacterial activity of these nanoparticles. It is suggested that antimicrobial activity increases as a function of nanoparticle concentration, based on in vitro tests on plates, but does not analyze the effect on biofilms.

In summary, although there are products aimed at preventing microbial growth in catheters for medical use, they are not demanded by highly complex public hospital establishments, due to their higher cost and the low effectiveness observed in clinical trials to reduce the incidence of CAUTI. Therefore, there is currently a need for a commercial catheter with antibiofilm activity to prevent such infections, which is particularly relevant for immunocompromised and critically ill patients, as they are the most at-risk groups.

### BRIEF DESCRIPTION OF THE FIGURES

FIG. 1. Methodology for the synthesis of the material of PEGylated copper nanoparticles embedded in silicone.

FIG. 2. UV-Vis spectra in kinetic experiments of copper ion release. (a) Elaboration of calibration curve with different concentrations and (b) different exposure times in samples of the material with an CuNP concentration of 2.2±0.10 pM.

FIG. 3. A: Determination of antimicrobial activity against E. coli ATCC 25922 at 2 incubation times, the % reduction in control (untreated silicone), PCS (CuNP·PEG₂₀₀₀) and CS (CuNP) is shown. B: Determination of the concentrations of CuNP-PEG₂₀₀₀ that confer antimicrobial activity to the material, where 4 different concentrations of nanoparticles were analyzed: 1. 2.2±0.10 pM, 2. 5.6±0.28 pM, 3. 12.1±0.89 pM, and 4. 13.8±0.81 pM.

FIG. 4. Determination of antibiofilm activity against E. coli and K. pneumoniae. Biofilm formation on C1, C2 and untreated silicone films (control) was evaluated in a continuous flow system after 24 h of incubation at 37° C. The biofilm formed was terminated using the eluted violet crystal staining method, which was quantified by absorbance at OD_{595 nm}. Statistical analyses were performed using the Mann-Whitney test. The statistical significance value for each film is shown.

FIG. 5. Representative scanning electron micrographs of silicone (A-B) and C1 material (B-C) implants extracted from murine bladders infected with E. coli at 96 h post-infection. Figures D and E correspond to implants in mice not infected with E. coli. The magnifications correspond to 16000× (A, C, and E) and 30,000× (B, D, and F).

FIG. 6. Bacterial load assessment. Catheter-associated urinary tract infection (CAUTI) was quantified at 96 hours. C57/Bl6 females 8-10 weeks old were inoculated with untreated catheters (silicone) and C2 catheters and infected with ≈1×10⁷ CFU of E. coli. Subsequently, Colony Forming Units (CFUs) were quantified in bladder and kidney homogenates.

FIG. 7. Cell viability assay. The fluorescence emitted at 590 nm (excl. 530 nm) by resazurin after 24 h of exposure of human HepG2 cells to C1 and C2 and untreated silicone (Si) was quantified. As a control, the cells were incubated only with DMEM medium, 10% fetal bovine serum and 1% antibiotics. No statistically significant differences were observed between the groups using the Mann-Whitney test.

### DETAILED DESCRIPTION OF THE INVENTION

The invention corresponds to a material based on silicone and copper nanoparticles (CuNP) coated with polyethylene glycol (PEG) that exhibits antimicrobial and antibiofilm activity, i.e. it allows for the reduction of the bacterial load and the formation of biofilm. The non-stick properties of PEG and the antimicrobial activity of copper act synergistically, generating resistance to protein adsorption and bacterial adhesion, and at the same time eliminate the bacterial load, resulting in a significant reduction in biofilm formation. In the case of medical uses, the material reduces the growth of microorganisms and the development of biofilms, and thus the risk of infection.

In a mouse model of catheter-associated urinary tract infection (CAUTI), it was determined that the intravesical implantation of a silicone catheter and copper nanoparticles (cNP) coated with polyethylene glycol (PEG) showed a bacterial load in the bladder and kidney compared to a silicone catheter. In addition, scanning electron microscopy showed less biofilm formation in the silicone catheter and copper nanoparticles (CuNP) coated with polyethylene glycol (PEG) compared to the silicone catheter. Both catheters produced a mild degree of inflammation when compared to a normal bladder.

A silicone material functionalized with PEG-coated copper nanoparticles with antimicrobial and antibiofilm activity is provided, comprising:
Activated surface silicone coated with a polyethylene glycol solution, where polyethylene glycol is selected from PEG silane, APTA, or N-silane. PEG-silane is preferentially selected from PEG₆₋₉ silane, PEG₉₋₁₂ silane, PEG₂₁₋₂₄ silane, and N-silane is N-6 silane; and
Copper nanoparticles (CuNP; with a diameter between 80-100 nm) with polyethylene glycol.
Silicone is biocompatible, chemically and thermally stable, and has a long service life. The silicone is chemically activated and then functionalized with a polyethylene glycol solution (PEGylating process).
Copper particles, and especially nanoparticles of this metal, have potent antimicrobial activity. CuNP have been shown to induce plasmid DNA degradation in a dose-dependent manner in Gram-positive and Gram-negative bacteria. Other studies have shown that smaller copper particle sizes promote antimicrobial activity due to their greater ability to enter the bacterial cytoplasm, resulting in increased intracellular oxidative stress. However, this same property must be taken into account to avoid adverse effects such as cytotoxicity and genotoxicity in humans. The developed material has an average CuNP diameter of 80-100 nm, and proved to be biocompatible and harmless, when evaluated in vitro in a human cell line.
An additional advantage of using CuNP as an antimicrobial agent compared to antibiotics is the lower generation of resistance.
PEG is a polyether with the formula H—(O—CH₂—CH₂)n-OH that is prepared by polymerization of ethylene oxide and is commercially available in a wide range of molecular masses from 300 g/mol to 10,000,000 g/mol. On the other hand, PEGylation refers to the process of covalent and non-covalent bonding or amalgamation of PEG polymer chains to molecules and macrostructures. It is important to mention that the biological and pharmacological characteristics of a PEGylated material will depend directly on the size and molecular weight of the PEG used. In the development of the invention, weights between 600 and 4000 g/mol were evaluated.
The CuNP were made from a precursor copper sulfate (CuSO₄·5H₂O), ascorbic acid (AA, 0.01 M) as a reducing agent, and PEG between 600 and 4000 g/mol as a stabilizing agent.
The PEGylated nPCNs obtained are incubated with silicone coated with polyethylene glycol, thus obtaining the silicone material with antimicrobial and antibiofilm activity according to the invention.

### Process of Elaboration of the Material of the Invention

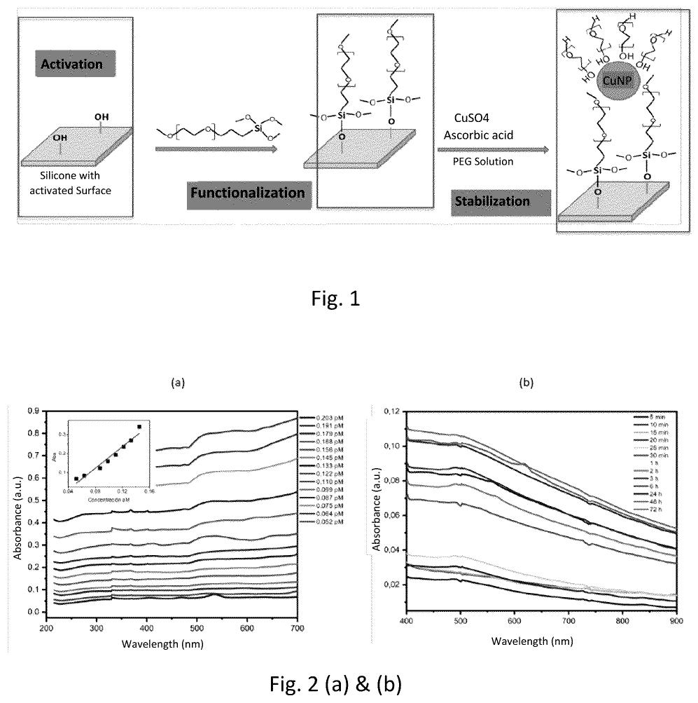

The silicone films to be used must be clean and their surface is activated, washed and dried, prior to functionalization with a polyethylene glycol solution (PEGylating process). The excess solution is removed and the material is washed and dried (FIG. 1).
Activation. The first stage involves providing the silicone material and activating the surface. For this, the silicone films were cleaned before use. Activation of the silicone surface was carried out by treatment with HCl, H₂SO₄/H₂O₂ (1:1) or a mixture of solvents (H₂O/HCl/H₂O₂) for 10 to 15 h at room temperature, resulting in a hydrophilic surface coated with silanol groups (Si—OH). Subsequently, the material is washed, subjected to sonication, dried with nitrogen and immediately immersed in the respective polyethylene glycol solution.
Functionalization. Functionalization is performed by immersing the silicone in a polyethylene glycol solution, preferably in a ratio of 1:5:5 to 1:10:10 of PEG-silane, acetic acid (0.1 M) and isopropyl alcohol respectively, for 10-15 h at room temperature.
Subsequently, the films are washed in ethanol and distilled water and then excess non-functionalized material is removed. The films were dried with nitrogen and stored dry under ambient conditions. The dry material is stored at room temperature.
Stabilization. PEGylated copper nanoparticles are made by adding CuSO₄·5H₂O, ascorbic acid and PEG solution.
Copper sulfate is mixed with PEG, the copper sulfate used was CuSO₄·5H₂O (0.01 M). A solution of ascorbic acid is prepared in water and NaOH, and then added over the solution of copper and PEG, stirring constantly.
After stirring, the precipitate is separated, preferably by centrifugation, by redispersing the nanoparticles in water. Optionally, the spin and redispersion steps in water are repeated until the excess PEG is removed.
The silicone surface previously functionalized with polyethylene glycol was incubated with the synthesized CuNP for a period of 10-15 h to promote chemical interaction between the polyethylene glycol chains present on the silicone surface with the CuNP.
The optimal concentration of CuNP during incubation is selected from: 2 to 14 pM; Preferably, it is selected from: 2.2±0.10 pM, 2. 5.6±0.28 pM, 3. 12.1±0.89 pM, and 4. 13.8±0.81 pM. In this way, the material of the invention of silicone functionalized with copper nanoparticles coated with PEG (PEGylated) is obtained; with activity that reduces the formation of biofilm.

### Advantages

The material, according to the invention, differs from other alternatives in that, in addition to conferring a powerful antimicrobial activity to the material granted by the presence of copper nanoparticles, it manages to prevent the adsorption of proteins on its surface, which in turn contributes to the prevention or reduction of biofilm formation. An additional benefit is that the use of copper as an active component decreases the likelihood of generating antimicrobial resistance. In addition, the materials made in accordance with the invention are not cytotoxic. The concentration of the components of the invention is such that it is the minimum that allows for obtaining the antimicrobial activity and preventing or reducing the formation of biofilm in the silicone material.

The material demonstrates a decrease in bacterial load in vivo, for example, when analyzing bladders and kidneys in the assays at 96 h post-infection. The decrease in bacterial count is an indicator of the antibacterial effect of the material according to the invention, by obtaining values of colony-forming units (CFUs) lower than those reported in the use of traditional silicone.

The material also demonstrated its antibiofilm activity, or ability to reduce bacterial colonization and the formation of a layer of bacteria on the surface of the material, when evaluated in a continuous flow system, with fluids containing bacteria and similar in composition to urine.

### Field of Application

Although the problem initially addressed was the development of urinary catheters that prevent the formation of biofilm, the material according to the invention allows the production of various products where silicone subjected to the same type of curing can be used.

Thus, it is possible to use the material in pharmaceutical, medical and food use, among others. Devices for medical use include those described in Table 1 and, in general, any device that can be made or coated with medical-grade silicone.

TABLE 1
Examples of devices that could be made
from the material of the invention.

| | |
|---|---|
| Silicone urinary catheter | Perperipherally inserted |
| Foley catheter | percutaneous catheter |
| Central venous catheter | Pig tail catheter |
| Tunneled central venous catheter | Silicone Arterial Line |
| Hickman central venous catheter | Continuous Irrigation Catheters |
| Subcutaneous catheter | Hemodialysis catheter |
| Peripheral venous catheter | Silicone tubes |
| Peripheral intravenous catheter | Silicone hoses |
| (intravenously) | Silicone ureteral stent |
| Silicone Face Masks | Silicone suction tubes |
| Silicone Fan Masks | Silicone nasogastric tubes |
| Silicone Implants | Silicone feeding tubes |
| Silicone prostheses: all | Silicone Drains: Jackson, Blake, |
| Silicone menstrual cups | Flat, Hemosuc/Hemovac |
| Silicone endotracheal tubes | Silicone Mechanical Fan Filters |
| Silicone laryngeal mask | Silicone Swan-Ganz |
| Silicone Anesthesia Mask | Silicone breath preservatives |
| Ambu (manual resuscitator) made of | Silicone Parenteral Nutrition Bags |
| silicone | Bags of silicone serums and blood |
| Silicone bags | products |
| Silicone Oxygen Masks | Silicone serum drops |
| Silicone patches | Silicone valves |
| Silicone Collectors | |

In the case of kitchen utensils, it is also possible to use the silicone material functionalized with copper nanoparticles according to the invention, as it is food-safe and safe for use in the kitchen.

In the case of the pharmaceutical industry, many patients do not take their medications as prescribed and need medical devices made of silicone to deliver controlled-release medications. The silicone material functionalized with copper nanoparticles, according to the invention, makes it possible to obtain new pharmaceutical-grade silicone materials that exhibit antibacterial and antibiofilm activity.

### EXAMPLES

### Example 1: Elaboration of the Silicone-Based Material and PEGylated Copper Nanoparticles for the Manufacture of Catheters

### Materials & Reagents

The silicone films (diameter 20 mm, thickness 0.024″) were supplied by Interstate IPS (USA). Deionized water was used to rinse the PDMS surfaces and prepare aqueous solutions. 2-[methoxy (polyethyleneoxy)-6-9-propyl]trimethoxysilane, 3-aminopropyltrimethoxysilane, N-(6-aminohexyl)aminopropyltrimethoxysilane were acquired from Gelest SA (USA). Hydrogen peroxide, copper (II) sulfate, sodium hydroxide, L-ascorbic acid, polyethylene glycol (PM g/mol: 2000, 3000, 4000) were purchased from Sigma-Aldrich.

Six materials were developed for prototype testing (see Table 2).

TABLE 2
Prototype formulations

| Formulación | PEG silano | PEG |
|---|---|---|
| 1 | PEG silano₆₋₉ | PEG₄₀₀₀ |
| 2 | PEG silano₆₋₉ | PEG₃₀₀₀ |
| 3 | PEG silano₆₋₉ | PEG₂₀₀₀ |
| 4 | APTA | PEG₃₀₀₀ |
| 5 | APTA | PEG₂₀₀₀ |
| 6 | N-6 amino | PEG₂₀₀₀ |

PEG silane6-9: 2-[methoxy(polyethyleneoxy)₆₋₉propyl]trimethoxysilane;
APTA: 3-aminopropyltrimethoxysilane;
N-6 amino: N-(6-aminohexyl)aminopropyltrimethoxysilane

The silicone films were cleaned by ethanol/water sonication (1:1, v/v) for 10 min before use. Activation of the silicone surface was carried out by treatment with HCl (30%) for 12 h at room temperature. The samples were washed 3 times in water, sonicated in water for 10 min, dried with nitrogen, and immediately immersed in the respective PEG-silane solution. Functionalization was performed using a 1:5:5 solution of PEG-silane, 0.1 M acetic acid and isopropyl alcohol, respectively, for 12 h at room temperature. Subsequently, the films were washed twice in ethanol and twice in water and then sonicated in water for 2 min to remove excess non-functionalized material. The films were dried with nitrogen and stored dry under ambient conditions.

The CuNP were obtained by chemical synthesis, using copper sulfate (CuSO₄*5H₂O), ascorbic acid (AA—0.01 M) as a reducing agent and PEG of different sizes (2000-3000-4000 g/mol) as a stabilizing agent.

A set of solutions was prepared by mixing 4.0 ml of CuSO₄·5H₂O (0.01 M) with 16.0 ml of PEG₂₀₀₀ at various concentrations. A stock solution of PEG₂₀₀₀ with a concentration of 0.5 M was prepared and various volumes of this stock solution (8, 4, 2 mL) were diluted to 16 mL with water, and it was these more dilute solutions that were added to the copper solution. In separate flasks, 0.5 ml of 0.1 M ascorbic acid was diluted in 10 ml of water and 3.0 ml of 0.5 M NaOH in 10 ml of water. Next, the ascorbic acid and sodium hydroxide solutions are mixed, and then added over the copper solution while agitating. The complete solution was kept in agitation for another 30 minutes.

After agitating, the solutions were centrifuged at 6000 rpm for 30 minutes. The precipitates were collected and redispersed in water. The obtained CuNP were centrifuged at the same rate for another 15 minutes, twice, to remove excess PEG.

The silicone surface previously functionalized with PEG-silane was incubated with synthesized CuNP for a period of 12 h to obtain the chemical interaction between the PEG-silane chains present on the silicone surface with the CuNP.

Subsequently, the physicochemical characterization of the prototype materials was carried out, which is relevant for the use of this material in the manufacture of medical devices.

FIG. 2 shows the release of copper ions from the nanoformulation of CuNP 2.2±0.10 pM, where the release of copper was determined by means of the dissolution-desorption mechanism, evidenced in the pseudo-isotherm adsorption experiment, which allows the maximum concentration of released copper to be obtained. It is observed that the maximum release of copper is obtained in the 72 hours, where on average a maximum of 0.074 pM and a minimum of 0.045 PM of CuNP are released.

### Example 2: Antimicrobial Capacity Assessment. Selection of the Best PEG-Silane Alternative and Concentration of CuNP

The evaluation of antimicrobial capacity was carried out with the formulation of the material Filme/PEG₆₋₉ silane/PEG₂₀₀₀+CuNP) with an CuNP size: 94.3±1.2 nm). Subsequently, the lowest necessary concentrations of CuNP were determined, obtaining conditions C1 and C2 (see Table 3):

TABLE 3
Characteristics of the evaluated material

| Condición | PEG-silano | PEG | CuNP |
|---|---|---|---|
| C1 | PEG silano₆₋₉ | PEG₂₀₀₀ | 2.2 ± 0.10 pM |
| C2 | PEG silano₆₋₉ | PEG₂₀₀₀ | 5.6 ± 0.28 pM |

### Antimicrobial Activity

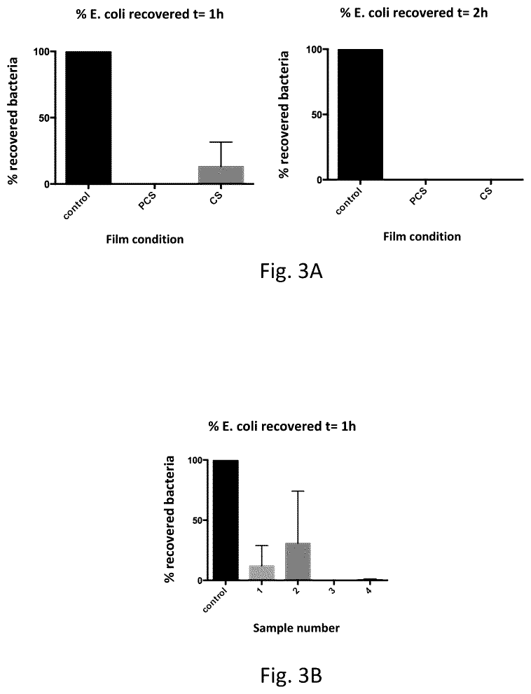

Antimicrobial activity against E. coli ATCC 25922 was determined at 2 incubation times in two types of samples. FIG. 3A shows the effect of the use of functionalized silicone, where the PCS material (CuNP·PEG₂₀₀₀) manages to reduce 100% of the bacterial load even at 1 hour of incubation at 37° C., versus the CS (CuNP) material that achieves the reduction after 2 hours.

The antimicrobial activity of four films with different concentrations of CuNP was also evaluated against a clinical isolate of urinary tract infection: E. coli ATCC 25922. FIG. 3B shows that two of the concentrations analysed manage to reduce 100% of the bacterial load.

In addition, the C1 formulation was evaluated for its ability to reduce bacteriuria and prevent bacteremia in a murine model of catheter-associated urinary tract infection (CFU≈1×10⁷ of E. coli), observing that E. coli biofilms do not occur in vivo in C1 material during the course of infection.

### Example 3: Assays of Antibacterial Activity Against E. coli, K. Pneumoniae, E. Faecalis and P. aeruginosa at Different Incubation Times

The following table compares the percentages of bacterial load reduction obtained for C1 (CuNP 2.2±0.10 pM) and C2 (CuNP 5.6±0.28 pM) materials at each incubation time and against the four bacteria used. In general, the two films showed similar antibacterial activities, obtaining a greater reduction of the bacterial load at 6 hours of incubation. For both materials, the percentages of reduction against E. coli and K. pneumoniae were greater than 90% in all the times evaluated, and against E. coli the reduction in some cases was even 100%. This is particularly important from an epidemiological point of view given that E. coli and K. pneumoniae are the most important etiological agents of CAUTI in Chile, the USA and Europe. In the case of E. faecalis and P. aeruginosa, the percentage reduction reached values greater than 80% in some cases.

These results demonstrate the influence of surface chemistry on interaction with bacteria, which in turn has a significant effect on the antimicrobial efficacy of CuNP surfaces. In that sense, increasing the release of copper ions in the bacteria/surface contact area on the invention material leads to greater efficiency in the elimination of bacteria.

TABLE 4
Reduction of bacterial load at different incubation times*.

| | | % Reduction | % Reduction |
|---|---|---|---|
| | | Average | Average |
| Bacteria | Time | C1 | C2 |
| E. coli | 2 h | 94.6 | 100 |
| | 3 h | 94.3 | 100 |
| | 4 h | 96.2 | 100 |
| | 6 h | 100 | 100 |
| K. pneumoniae | 2 h | 75.2 | 75.1 |
| | 3 h | 97.5 | 75.2 |
| | 4 h | 87.1 | 74.8 |
| | 6 h | 99.8 | 93.2 |
| E. faecalis | 2 h | 69.4 | 45.0 |
| | 3 h | 59.5 | 76.6 |
| | 4 h | 55.8 | 48.8 |
| | 6 h | 77.1 | 80.9 |
| P. aeruginosa | 2 h | 69.0 | 71.4 |
| | 3 h | 66.7 | 85.0 |
| | 4 h | 68.3 | 71.4 |
| | 6 h | 95.8 | 83.6 |

*Reduction in the number of bacteria recovered from control silicone films. The average of two trials performed in duplicate is shown.
As Table 4 shows, C1 and C2 films reduce the bacterial load of E. coli, K. pneumoniae, E. faecalis and P. aeruginosa compared to silicone films.

### Example 4: In Vitro-Continuous Flow System to Evaluate Biofilm Formation

The biofilm formation capacity of the prototype material C1 (CuNP 2.2±0.10 pM) and C2 (CuNP 5.6±0.28 pM) was determined in a continuous flow system, using the uropathogenic E. coli strain ATCC 25922 and K. pneumoniae ATCC 700603.

Continuous flow system: A preinoculum was loaded at 37° C. of 10 mL of LB with K. pneumoniae, or of LB supplemented with Amp for E. coli ATCC 25922 (transformed with the pDiGc plasmid). The next day, the films (C1, C2, Si control) were sterilized for 30 min (15 min per side) with UV radiation under a hood. The entire flow-through system was also sterilized separately. Sterile films were duplicated in 4 chambers of the system (2 films each) under sterile conditions. A continuous flow rate of 3.3 mL/min was set for 24 h at 37° C.

Biofilm quantification: After incubation for 24 h at 37° C., the films were washed with 4 mL PBS and then fixed with 70% methanol for 7 min (7). Subsequently, the methanol was extracted, the films were left to dry and incubated with 0.5% violet crystal for 15 min. Then 3×10 mL were washed with distilled water. To quantify staining, the films were immersed in 1 mL of 33% acetic acid, vortexed for 20 s, and quantified at OD 595 nm of solution. Tests were carried out in duplicate for each prototype.

Statistical analysis: The Mann-Whitney test was used to determine significant differences in the formation of E. coli or K. pneumoniae biofilms in C1 or C2 films compared to control films (silicone).

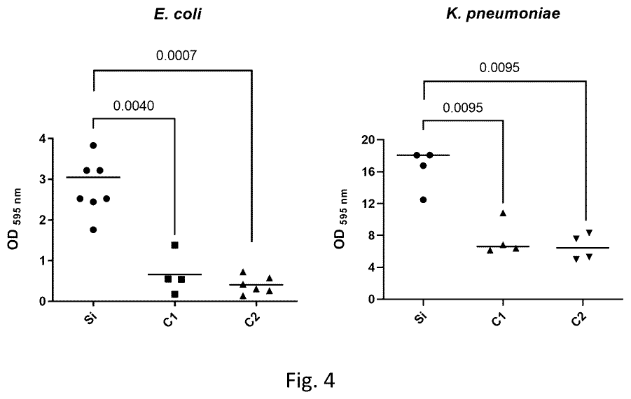

Results: The evaluation time for biofilm formation was 24 h in AUM medium. As expected, the bacteria evaluated formed a biofilm on the control silicone surface. Notably, K. pneumoniae generated 6 times more biofilm compared to E. coli, which is similar to what has been reported in other studies evaluating biofilm production in these bacteria. Notably, C1 and C2 films significantly reduced (p<0.05) biofilm formation for both E. coli and K. pneumoniae (FIG. 4). In particular, for C1, the reduction of biofilm against E. coli and K. pneumoniae was 5.2 and 2.7 times, respectively. Similarly, for C2, the reduction in biofilm formation against E. coli and K. pneumoniae was 7.9 and 2.8 times, respectively.

No statistical differences were observed in the reduction of biofilm formation between C1 and C2. Therefore, C1 and C2 films significantly reduce the formation of biofilms generated by E. coli ATCC 25922 and K. pneumoniae ATCC 700603 compared to silicone films.

### Example 5: In Vivo-Experiments in Murine Model of CAUTI (Catheter-Associated Urinary Tract Infection)

Female C57BL/6 mice weighing 26-35 g, 8-10 weeks old were divided into 2 groups, group C1 and control group (5 animals/group). A piece of 7 mm polyethylene tubing (PE10 BD cat No. 427400) was placed on a 30 G×½ sterile metal needle (0.3 mm×13 mm BD Precision Glide) followed by a 5 mm segment of C1 prototype material or the control. The needle was placed into the urethral opening and the tube was moved over the needle until the material segment of the invention or control was deposited inside the bladder. The needle and the 7 mm tube were later removed.

The mice were infected immediately after insertion of the silicone segment, where the inoculum was administered in 50 μL of 1×PBS at a rate of 10 μL/s, using a tuberculin syringe. The number of CFUs present in the inoculum corresponded to ˜ 1×10⁷ CFU of E. coli.

### Evaluation of Biofilm Formation.

The devices (C1 and silicone) retrieved from the bladders were fixed in 2.5% glutaraldehyde and prepared for scanning electron microscopy (SEM) to evaluate biofilm formation.

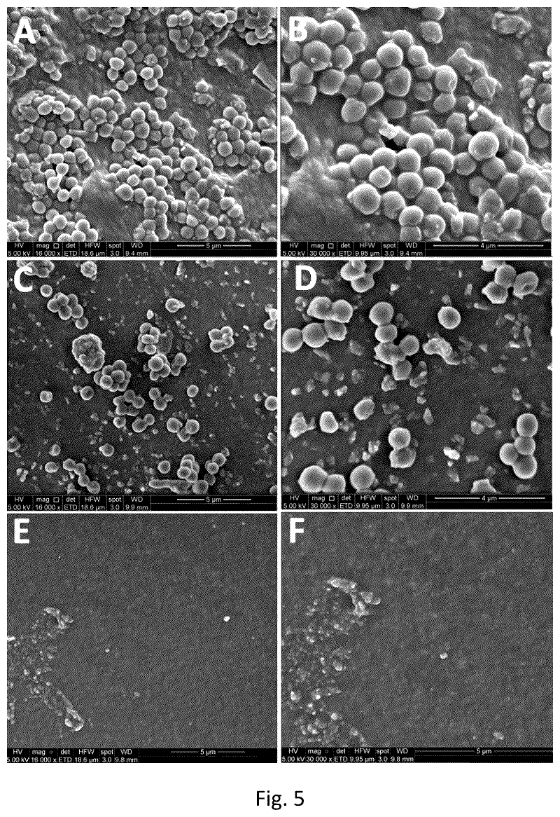

The control implants (silicone) of the infected mice were coated with bacteria in the lumen that are observed embedded in what appears to be an extracellular matrix (FIGS. 5a and b), unlike implant C1 in which bacteria are observed in the absence of biofilm (FIGS. 5c and d). The mice with both implants, but not infected, did not present bacteria in the implants, although it is possible to think that the implants may be coated with host factors present in the urine (FIG. 5 e and f).

### Bacterial Load Assessment

The mice were euthanized 96 hours after infection and subsequently the bladders and kidneys were removed, homogenized and diluted in PBS and platelet in LB/ampicillin agar media in dilutions 10⁻¹ to 10⁻⁸. The bacterial load was determined after incubation for 24 to 48 h at 37° C. and was expressed as total CFU per organ.

Statistical analysis: The Student's T test was used to determine significant differences in the number of colony-forming units (CFUs) of E. coli in bladder and kidney homogenates of mice with C1 catheter compared to controls (silicone catheter).

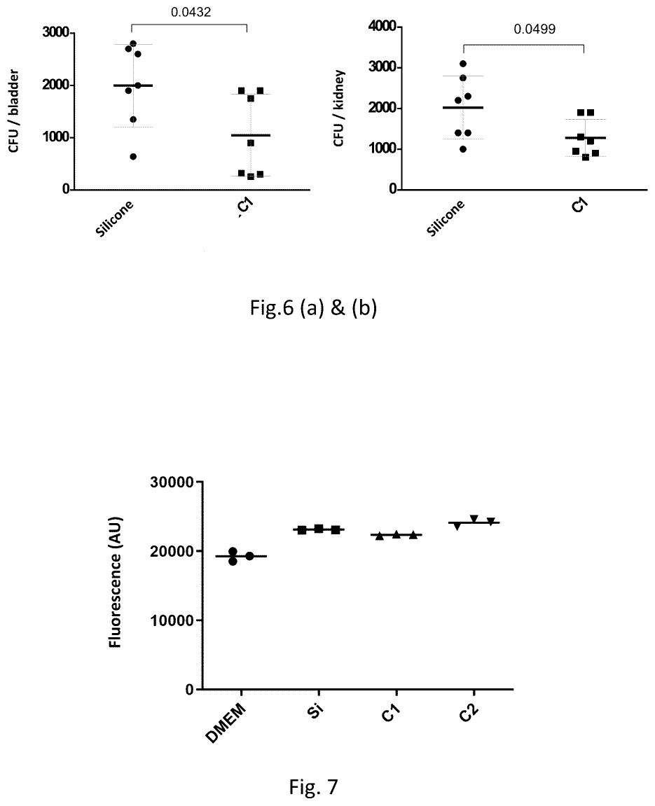

Results: In animals with C1 implants, E. coli bacteria reached an average titer of 1.05×10³ CFU/ml at 96 hours in the bladders, which was significantly lower (P=0.432) than the bacterial titers (2×10³ CFU/ml) recovered from the bladders of the control implant animals (FIG. 6a). A similar result was observed for kidneys, where C1-implanted mice achieved an average of 1.3×10³ CFU/mL, which was significantly lower (P=0.499) than bacterial titers (2×10³ CFU/mL) retrieved from the kidneys of control implant animals (FIG. 6b).

Inflammation evaluation. For histological analyses, the bladders were fixed in buffered formaldehyde for 2 hours and dehydrated in 70% ethanol overnight at 4° C. They were then embedded in paraffin, sectioned, and stained with hematoxylin and eosin (H&E) for light microscopy.

Results: The implantation of catheters in the bladder and infection with E. coli caused histological changes in the bladder at the epithelium, submucosa and muscle levels. The changes were mild (1 out of 3). Although there are no significant differences in the degrees of inflammation between the C1 implanted and control animals, it was observed that in the control animals a mild, perivascular, chronic lymphocytic to neutrophilic cystitis was observed, with mild edema and mild epithelial changes. compared to the mild, perivascular, chronic lymphocytic cystitis observed in C1 implanted animals.

### Example 6: In Vitro Evaluation of Cytotoxicity of the Material in Hep-G2 Cells

The HepG2 cell line of hepatocellular carcinoma (passage 19) was used for the assays. The cells were incubated in Dulbecco's Modified Eagle Medium (DMEM) with 10% fetal bovine serum (FBS) and 1% antibiotic (penicillin and streptomycin). The crops were kept at 37° C. with an atmosphere humidified with 5% CO₂.

The “In vitro toxicology assay, Resazurin based” kit (Sigma-Aldrich, Darmstadt, Germany; Cat No. TOX8-1KT).

FIG. 7 shows the results of the quantification of the fluorescence emitted by resazurin, which accounts for active cellular metabolism. The observed results showed that the alternative hypothesis for a p<0.05 is rejected and the means of each experimental condition are similar, therefore, there is no significant difference in the cells that were exposed to the medium with the C1 or C2 material compared to the control without films or with unmodified silicone films (Si). Therefore, the materials made according to the invention are not cytotoxic to eukaryotic cells under the conditions studied.

### Example 7: Properties of the Material Subjected to Conditions of Use

Evaluation of physical, antimicrobial and antibiofilm properties of material subjected to UV sterilization and autoclave.

The samples were subjected to UV irradiation sterilization processes in the in vitro phase. As a result, the materials showed antimicrobial capacity.

1-14. (canceled)

15. A silicone material with activity that reduces the formation of biofilm, comprising:
a) Chemically activated surface silicone;
b) Coating selected from PEG, APTA or N-6 silane, where PEG is selected from PEG₆₋₉ silane, PEG₉₋₁₂ silane, PEG₂₁₋₂₄ silane;
c) Copper nanoparticles with a medium diameter of 80-100 nm at a concentration between 2 and 14 pM; and
d) Polyethylene glycol from 600 to 4000 g/mol,
where the silicone according to a) is functionalized with the coating according to b), where the nanoparticles described in c) are PEGylated with the polyethylene glycol described in d) and where these PEGylated nanoparticles in turn form a coating on the functionalized silicone.

16. The material according to claim 15, wherein the size of copper nanoparticles is between 90 to 100 nm.

17. The material according to claim 15, wherein the size of copper nanoparticles is between 80-90 nm.

18. The material according to claim 15, wherein the concentration of copper nanoparticles is selected from between 2.2±0.10, 5.6±0.28, 12.1±0.89 and 13.8±0.81 pM.

19. The material according to claim 1, wherein the molecular weight of polyethylene glycol is between 1000 to 3000 g/mol.

20. The material according to claim 4, wherein the molecular weight of polyethylene glycol is between 2000 to 3000 g/mol.

21. The material according to claim 1, wherein the polyethylene glycol coating is at least one selected from PEG₆₋₉ silane and PEG₉₋₁₂ silane.

22. The material according to claim 1, wherein the surface of the silicone is activated with HCl, H₂SO₄/H₂O₂ (1:1) or a mixture of solvents (H₂O/HCl/H₂O₂).

23. A process for making a silicone material with anti-biofilm activity, comprising:
i) Chemically activating the surface of the silicone;
ii) Functionalizing the silicone surface by coating it with PEG, APTA or N-6 silane, where PEG is selected from PEG₆₋₉ silane, PEG₉₋₁₂ silane, or PEG₂₁₋₂₄ silane;
iii) Synthesizing the PEGylation of copper nanoparticles with a medium diameter of 80-100 nm with polyethylene glycol of 600 to 4000 g/mol; and
iv) Incubating the functionalized silicone with the PEGylated copper nanoparticles.

24. The process according to claim 23, wherein prior to activation, the surface of the silicone is previously washed with an ethanol/water solution.

25. The process according to claim 23, wherein activation is performed with HCl, H₂SO₄/H₂O₂ (1:1) or a mixture of solvents (H₂O/HCl/H₂O₂).

26. The process in accordance with claim 23, wherein activation is preferably performed with HCl.

27. The process according to claim 23, wherein after functionalization, the material is subjected to alcohol and water washing, sonication and drying.

28. The process according to claim 23, wherein the weight of stage iii) polyethylene glycol is between 1000 to 3000 g/mol.
